SEQUENCE LISTING (40 sequences)

SEQ ID NO: 1 - DNA, 101 bp, Daucus carota
tgtttgagactcwactcttctcctttaccaacgaaagtatttatttgtgagcaatctttcatgtcaaacctagcaagtaccttctcaatatggctctttta

SEQ ID NO: 2 - DNA, 101 bp, Daucus carota
tgtttgagactcwactcttctcctttaccaacgaaagtatttatttgtgaacaatctttcatgtcaaacctagcaagtaccttctcaatatggctctttta

SEQ ID NO: 3 - DNA, 71 bp, Daucus carota
tcctatctctcttcctcctcacaatccttcccattctccactccttaccagccataccagacccaaccaca

SEQ ID NO: 4 - DNA, 71 bp, Daucus carota
tcctatctctcttcctcctcacaatccttcccattctacactccttaccagccataccagacccaaccaca

SEQ ID NO: 5 - DNA, 101 bp, Daucus carota
attatacttatacttgtttaaatatgtggtaactttgggtgccaatcactagagcaaatttttatgtaaaagttgtcaaaaatacaaacaaagrgagagaa

SEQ ID NO: 6 - DNA, 101 bp, Daucus carota
attatacttatacttgtttaaatatgtggtaactttgggtgccaatcactggagcaaatttttatgtaaaagttgtcaaaaatacaaacaaagrgagagaa

SEQ ID NO: 7 - DNA, 101 bp, Daucus carota
cttcacaactatttachaagtaatttaaaacttctttaatctgtgaagctcgatatcttattgctttaactcctataacccgacwttctcaacatatttct

SEQ ID NO: 8 - DNA, 101 bp, Daucus carota
cttcacaactatttachaagtaatttaaaacttctttaatctgtgaagcttgatatcttattgctttaactcctataacccgacwttctcaacatatttct

SEQ ID NO: 9 - DNA, 71 bp, Daucus carota
cgaccaatgatatcagttttttcgacgataataatctcgggtcgttagaattggattctctctggacttga

SEQ ID NO: 10 - DNA, 71 bp, Daucus carota
cgaccaatgatatcagttttttcgacgataataatcttgggtcgttagaattggattctctctggacttga

SEQ ID NO: 11 - DNA, 101 bp, Daucus carota
ttatatactgtataaaacaatgattagtgcaacaaaaattagtgatagtcatagttataaatactggtaaggaaactagtttttagtttgtatttaagagt

SEQ ID NO: 12 - DNA, 101 bp, Daucus carota
ttatatactgtataaaacaatgattagtgcaacaaaaattagtgatagtcgtagttataaatactggtaaggaaactagtttttagtttgtatttaagagt

SEQ ID NO: 13 - DNA, 71 bp, Daucus carota
atccgtcaattagaaaaagttaaaaaacactctctctagacacccattctcatttgaagcaacacgtgatg

SEQ ID NO: 14 - DNA, 71 bp, Daucus carota
atccgtcaattagaaaaagttaaaaaacactctctctggacacccattctcatttgaagcaacacgtgatg

SEQ ID NO: 15 - DNA, 101 bp, Daucus carota
gctacaacttttatctttttcagtctttacatgaagtamtagccgtaaattttkcaagccaaaggtgcaacacattgtagactcgacaatatctttcactc

SEQ ID NO: 16 - DNA, 101 bp, Daucus carota
gctacaacttttatctttttcagtctttacatgaagtamtagccgtaaatcttkcaagccaaaggtgcaacacattgtagactcgacaatatctttcactc

SEQ ID NO: 17 - DNA, 101 bp, Daucus carota
cacttgtcctgaagaatgtcaaggcaaatattcccatactggtcaacatttggatggaagcacattgtctcaaacttcacttggggaggcttgaaaggata

SEQ ID NO: 18 - DNA, 101 bp, Daucus carota
cacttgtcctgaagaatgtcaaggcaaatattcccatactggtcaacattgggatggaagcacattgtctcaaacttcacttggggaggcttgaaaggata

SEQ ID NO: 19 - DNA, 101 bp, Daucus carota
taaggcttgcrttggmttaaaragttgcagcatcgacgtatcagtgtcaacttttgggaatccgtgtagaggagttacaaagagtttagcagtagaagcwt

SEQ ID NO: 20 - DNA, 101 bp, Daucus carota
taaggcttgcrttggmttaaaragttgcagcatcgacgtatcagtgtcaatttttgggaatccgtgtagaggagttacaaagagtttagcagtagaagcwt

SEQ ID NO: 21 - DNA, 101 bp, Daucus carota
atgacaccaacacggtgaaagtcagtgtatctggccttttggaggtggatttgaaggtgactccaataaaagaaaaggaaaacaaggtgcacaactaccag

SEQ ID NO: 22 - DNA, 101 bp, Daucus carota
atgacaccaacacggtgaaagtcagtgtatctggccttttggaggtggatgtgaaggtgactccaataaaagaaaaggaaaacaaggtgcacaactaccag

SEQ ID NO: 23 - DNA, 71 bp, Daucus carota
ccatacccattcttttgaaatgaaaatgcaaaacagaagtcttccagtagctctctacctgcatttgacaa

SEQ ID NO: 24 - DNA, 71 bp, Daucus carota
ccatacccattcttttgaaatgaaaatgcaaaacagaggtcttccagtagctctctacctgcatttgacaa

SEQ ID NO: 25 - DNA, 71 bp, Daucus carota
aaacattaaacttggcggatgatgatatatataatgccgtgctacttgttgcagctgcttataatcacttt

SEQ ID NO: 26 - DNA, 71 bp, Daucus carota
aaacattaaacttggcggatgatgatatatataatgcggtgctacttgttgcagctgcttataatcacttt

SEQ ID NO: 27 - DNA, 71 bp, Daucus carota
gatcgaagggggtgcggccagaggtctttgcccgagcgtatgcccgtgtcttctcctcgttacttgtcctt

SEQ ID NO: 28 - DNA, 71 bp, Daucus carota
gatcgaagggggtgcggccagaggtctttgcccgagcatatgcccgtgtcttctcctcgttacttgtcctt

SEQ ID NO: 29 - DNA, 71 bp, Daucus carota
ctctcggracccaaccaattaccctacgaactttctcctcgaagagtactacacatgtttttgctgcctca

SEQ ID NO: 30 - DNA, 71 bp, Daucus carota
ctctcggracccaaccaattaccctacgaactttctcatcgaagagtactacacatgtttttgctgcctca

SEQ ID NO: 31 - DNA, 71 bp, Daucus carota
gagaatctgccgtacggcgtgttcaagcctgacgaatgttcggcacctcgacctggcgttgctatcggrga

SEQ ID NO: 32 - DNA, 71 bp, Daucus carota
gagaatctgccgtacggcgtgttcaagcctgacgaatcttcggcacctcgacctggcgttgctatcggrga

SEQ ID NO: 33 - DNA, 71 bp, Daucus carota
gccaaaggctactacaaagtggttgcaagtttcaaaattgatgcacaagggtgctgtgtcaagtgaagatc

SEQ ID NO: 34 - DNA, 71 bp, Daucus carota
gccaaaggctactacaaagtggttgcaagtttcaaaactgatgcacaagggtgctgtgtcaagtgaagatc

SEQ ID NO: 35 - DNA, 71 bp, Daucus carota
gaaaatatcatcgccacgagataatttttgctgatcaacaaagacttgagagtaacattcgacttcccgtg

SEQ ID NO: 36 - DNA, 71 bp, Daucus carota
gaaaatatcatcgccacgagataatttttgctgatcagcaaagacttgagagtaacattcgacttcccgtg

SEQ ID NO: 37 - DNA, 71 bp, Daucus carota
ttttacatagttacaattagaaattcagatcccaccttttagactccaacctctccggctaacttgaaagg

SEQ ID NO: 38 - DNA, 71 bp, Daucus carota
ttttacatagttacaattagaaattcagatcccacctcttagactccaacctctccggctaacttgaaagg

SEQ ID NO: 39 - DNA, 71 bp, Daucus carota
tacaactcgatgcttatatatcacgaaaagaactcttcaaggcagaagcaaaatattggaatgaaataata

SEQ ID NO: 40 - DNA, 71 bp, Daucus carota
tacaactcgatgcttatatatcacgaaaagaactctttaaggcagaagcaaaatattggaatgaaataata

(19) United States
(12) Patent Application Publication
(10) Pub. No.: US 2024/0381823 A1
(43) Pub. Date: Nov. 21, 2024

(54) Tropical Root-Knot Nematode Resistant Carrot Plant

(71) Applicant: Bejo Zaden B.V., Warmenhuizen (NL)

(72) Inventors: Adriana Dorien Haarsma, Warmenhuizen (NL); Diana Katschnig, Warmenhuizen (NL); Willem Arie Zwaan, Warmenhuizen (NL); Peter Arnoldus Dekker, Warmenhuizen (NL); Albertus Johannes Maria Schrijver, Warmenhuizen (NL)

(21) Appl. No.: 18/694,470

(22) PCT Filed: Sep. 23, 2021
(86) PCT No.: PCT/EP2021/076245

§ 371 Date: Mar. 22, 2024

(51) Int. Cl. A01H 6/06; A01H 1/00; A01H 5/06; C12N 15/82; C12Q 1/6895

(52) U.S. Cl. CPC A01H 6/068; A01H 1/1265; A01H 5/06; C12N 15/8205; C12Q 1/6895

(57) ABSTRACT

The present invention relates to tropical root-knot nematode resistant carrot plants comprising a first tropical root-knot nematode resistance providing genomic fragment. The present invention further relates to methods for identifying tropical root-knot nematode resistant carrot plants, methods for providing tropical root-knot nematode resistant carrot plants and means for identifying tropical root-knot nematode resistant carrot plants.

### CROSS-REFERENCE TO RELATED APPLICATION

This application is the United States national phase of International Application No. PCT/EP2021/076245 filed Sep. 23, 2021, the disclosure of which is hereby incorporated by reference in its entirety.

### REFERENCE TO A SEQUENCE LISTING

The Sequence Listing associated with this application is filed in electronic format via Patent Center and is hereby incorporated by reference into the specification in its entirety. The name of the text file containing the Sequence Listing is 2401492_ST25.txt. The size of the file is 9,330 bytes, and the file was created on Mar. 8, 2024.

### BACKGROUND OF THE INVENTION

### Field of the Invention

The present invention relates to carrot plants with one or two tropical root-knot nematode resistance providing genomic fragments. The present invention further relates to methods for identifying tropical root-knot nematode resistant carrot plants, methods for providing tropical root-knot nematode resistant carrot plants and means for identifying tropical root-knot nematode resistant carrot plants. The present invention also relates to seeds, plant parts, and especially edible plant parts of the present plants.

Carrot, or Daucus carota, is a cultivated plant from the Umbelliferae (or Apiaceae) family, which is common in many parts of the world. The Umbelliferae family encompasses more than 3,500 species, including next to the genus Daucus, various other cultivated plants, for example, caraway, celery, coriander, dill, fennel, parsley, and parsnip.

Wild carrot, Daucus carota L., is endemic to large parts of the world. It has a white taproot that is initially edible but becomes woody after prolonged growth. The cultivated carrot, Daucus carota, and especially Daucus carota ssp. sativus is a root vegetable that is usually orange, but purple, red, yellow, and white varieties are also known.

In moderate climate zones, Daucus carota is a biennial plant that grows vegetatively in the first year after sowing. After overwintering, the plant will flower in the second year of cultivation. In tropical and subtropical areas, the carrot plant has an annual life cycle, and the shift from vegetative to generative growth occurs without vernalization.

Two types of male sterility are described in the genus Daucus. In the so-called brown anther type, anthers degenerate and shrivel before they can spread pollen. In the petaloid type, the stamens are replaced by petal-like structures.

Male sterility observed in cultivated carrots is generally due to cytoplasmic male sterility caused by mitochondrial defects. Since mitochondria are transferred to the offspring by egg cells only, this trait is maternally inherited. Male sterility is a useful trait in carrot breeding as it enables 100% cross-pollination. As a result, hybrids of Daucus are readily produced. Moreover, heterosis, or hybrid vigour, can be very strong in carrot.

Carrot is cultivated for its nutritious taproot. A major part of this root consists of an outer phloem cortex and an inner xylem core. Moreover, a large proportion of the cortex relative to the core is considered to have high horticultural quality. The taproot is rich in carotene, especially β-carotene, an important antioxidant that can be metabolized to vitamin A. Carrots are also a source of dietary fibre, vitamins C, B6, and K, and the antioxidant falcarinol. Antioxidants (including carotenoids) have been studied for their ability to prevent chronic disease.

The root length of carrot plants varies from 5 up to 40 cm, while the diameter can vary between 1 to 10 cm. Taproots also come in various shapes. Round, conical, or more cylindrical shapes are preferred depending on the purpose. The taproot of wild varieties is white, but cultivated carrots are ordinarily orange, sometimes red, purple, black, or yellow.

Cultivation of carrots occurs globally. In 2011, more than 35 million tons of carrots were produced. Yet, many pests are known that threaten harvests around the globe. These include bacterial, fungal, viral, and viroid diseases, but also insect and nematode pests. Major bacterial and fungal diseases are caused by, among others, Xanthomonas hortorum, Erwinia carotovora, Alternaria dauci, Alternaria radicina, Pythium spp., Rhizoctonia spp., Sclerotinia spp., Fusarium spp, Botrytis cinerea, and Phytophthora spp. Nematodes, such as Heterodera carotae, Meloidogyne spp. and Pratylenchus spp., cause severe damage to the taproot, resulting in yield loss and a product unsuitable for market. These yield losses have led to dedicated breeding programs by companies and governmental institutes to introduce genetic resistances in carrot plants against these various pathogens.

Root-knot nematodes of the genus Meloidogyne are examples of pests that significantly impact carrot cultivation. Root-knot nematodes cause an estimated annual loss of up to roughly $157 billion. These obligate plant-parasites have a worldwide distribution and a broad host range, including many important vegetable crops, particularly tomato, aubergine, okra, cucumber, melon, carrot, gourds, lettuce, and peppers. The genus Meloidogyne includes more than 90 species, but Meloidogyne arenaria, Meloidogyne javanica, Meloidogyne incognita, and Meloidogyne hapla are considered the economically most important species. The species Meloidogyne hapla occurs predominantly in cooler climates, and Meloidogyne arenaria, Meloidogyne javanica and Meloidogyne incognita, are present in warmer regions, in particular areas with tropical and sub-tropical climates and are referred to as tropical root-knot nematodes.

Once the carrot plant is infested with tropical root-knot nematodes, numerous galls form on the taproot and secondary roots. Galls are abnormal outgrowths of plant tissues. The taproot of the carrot plant can become forked, and the growth of the carrot plant stunted. The galling and forking of the storage root leads to a malformed carrot, which, although still edible, is unacceptable for market. The amount of galling and forking is directly linked to nematode pressure and is worsened by warmer weather when the nematodes are more active. Yield and market losses can be significant. For example, losses of up to 50% have been reported in southern regions of India.

### Description of Related Art

The use of soil-applied nematicides is one effective approach to preventing Meloidogyne spp. infections. However, this approach is laborious and expensive. Moreover, the use of pesticides is increasingly restrained and organically grown vegetables are becoming increasingly popular.

Crop rotations and non-chemical management, including biological control organisms, can only control tropical root-knot nematodes to a limited extent, due to the wide host range of the nematodes, which includes many weeds. As a result, rotation options are limited.

The use of genetically resistant carrot varieties is the most efficient manner to prevent infection by tropical root-knot nematodes. Several resistances to Meloidogyne javanica and Meloidogyne incognita in carrot plants have been described in the prior art.

For example, two resistance loci that provide resistance to Meloidogyne javanica have been mapped to chromosome 8. The first locus was designated the Mj-1 locus and was discovered in the carrot cultivar Brasilia, in particular the line Br1252. The resistance provided by the Mj-1 locus is nearly completely dominant. The locus also provides partial resistance to Meloidogyne incognita. The second locus was designated Mj-2 and was discovered in the Chinese cultivar Ping Ding, also known as PI 652188. The Mj-2 locus has an incompletely dominant pattern of inheritance. Preliminary results suggest that this locus may also provide some resistance to Meloidogyne incognita.

In addition, Parsons et al. 2014 mapped resistance to Meloidogyne incognita in various carrot cultivars, in particular Brasilia from Brazil (Br1091), Homs from Syria, and the European cultivar Scarlet Fancy x Favourite (SFF). Resistance loci were identified on chromosomes 1, 2, 4, 8, and 9 in these cultivars (see Table 1). These loci are quantitative trait loci (QTLs) that each contribute small amounts to the overall resistance of the plant. As a result, it is necessary to combine several QTLs to provide carrots with an effective resistance against Meloidogyne incognita and Meloidogyne javanica. Combining these multiple resistance loci using breeding, however, is challenging and therefore, time-consuming and unpractical.

TABLE 1
Chromosomal location of previously reported QTLs conferring
resistance to Meloidogyne incognita or Meloidogyne javanica.
No QTLs have been described that confer
resistance to Meloidogyne arenaria.

| Source | Chr | Pos. (cM) | 1.5 LoD | Remarks |
|---|---|---|---|---|
| Brasilia | 1 | 67 | 52-75 | |
| | 8 | 42 | 42-56 | Co-localizes |
| | | | | with Mj-1 locus |
| Homs | 1 | 35 | 23-65 | |
| | 2 | 43 | 4-66 | |
| | 2 | 63 | 61-67 | |
| | 8 | 42 | 41-44 | Co-localizes |
| | | | | with Mj-1 locus |
| | 9 | 4 | 4-22 | |
| | 9 | 10 | 4-13 | |
| SFF | 4 | 33 | 15-57 | |
| | 8 | 42 | 27-59 | Co-localizes with Mj-1 |

Explanation of abbreviations used: Chr=chromosome; Pos. is the genomic position in centimorgan (cM); and 1.5 LoD is the 1.5-LOD support interval (the region where the LOD score is within 1.5 of its maximum) and indicates the most plausible location for the QTL

Hence, there is a need for a new genomically encoded resistance gene that confers resistance to the tropical root-knot nematode species Meloidogyne incognita as a monogenic dominant inherited trait. Preferably, such a resistance gene would confer resistance to not just Meloidogyne incognita, but to multiple tropical root-knot nematodes species, in particular Meloidogyne arenaria and Meloidogyne incognita as such a genomically encoded resistance could easily be combined with resistances to Meloidogyne javanica known in the prior art to yield a carrot plant resistant to all three tropical root-knot nematode species. Most preferably, a new genomically encoded resistance gene confers resistance to all three tropical root-knot nematode species, i.e., Meloidogyne incognita, Meloidogyne arenaria and Meloidogyne javanica.

### SUMMARY OF THE INVENTION

Considering the above, it is an object of the present invention amongst other objects, to provide carrot plants, or Daucus carota plants, resistant to the tropical root-knot nematode species Meloidogyne incognita; more preferably, to the tropical root-knot nematode species Meloidogyne incognita and Meloidogyne arenaria; and most preferably, to all three tropical root-knot nematodes Meloidogyne incognita, Meloidogyne arenaria and Meloidogyne javanica.

The present invention meets the above object, amongst other objects, as outlined in the appended claims.

### DESCRIPTION OF THE INVENTION

Specifically, this object, amongst other objects, is achieved, according to a first aspect, by providing a carrot plant resistant to tropical root-knot nematodes, comprising on chromosome 4 of said carrot plant a first tropical root-knot nematode resistance providing genomic fragment, wherein said first genomic fragment comprises a nucleic acid sequence selected from the group consisting of SEQ ID No. 1, SEQ ID No. 3, SEQ ID No. 5, SEQ ID No.7, SEQ ID No. 9, SEQ ID No. 11, SEQ ID No. 13, SEQ ID No. 15, SEQ ID No. 17, SEQ ID No. 19, SEQ ID No. 21 and SEQ ID No. 23. The indefinite article “a” or “an” can be used interchangeably with the phrases “one or more” or “at least one”. As such, the word “a” should be understood to mean one, two, three, four, five, six, or more, or all. Preferably, the first tropical root-knot nematode resistance providing genomic fragment on chromosome 4 comprises SEQ ID No. 9; more preferably SEQ ID No. 9 and SEQ ID No. 11; more preferably SEQ ID No. 5, SEQ ID No. 7, SEQ ID No. 9 and SEQ ID No. 11; even more preferably SEQ ID No. 1, SEQ ID No. 3, SEQ ID No. 5, SEQ ID No. 7, SEQ ID No. 9 and SEQ ID No. 11; yet even more preferably SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19 and SEQ ID No. 21; and most preferably SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, and SEQ ID No. 23. The first tropical root-knot resistance providing genomic fragment is not comprised of SEQ ID No. 2, SEQ ID No. 4, SEQ ID No. 6, SEQ, ID No. 8, SEQ ID No. 10, SEQ ID No. 12, SEQ ID No. 14, SEQ ID No. 16, SEQ ID No. 18, SEQ ID No. 20, SEQ ID No. 22, and SEQ ID No. 24.

Tropical root-knot nematode is defined as a group consisting of three tropical root-knot nematode species: Meloidogyne incognita, Meloidogyne arenaria and Meloidogyne javanica; preferably, Meloidogyne incognita and Meloidogyne arenaria; most preferably, Meloidogyne incognita.

Carrot plant means a plant of the species Daucus carota; preferably, a cultivated carrot plant; most preferably, a commercially cultivated carrot plant.

Alternatively, this object, amongst other objects, is achieved by providing a tropical root-knot nematode resistant carrot plant comprising two tropical root-knot nematode resistance providing genomic fragments, wherein a first tropical root-knot resistance providing genomic fragment is located on chromosome 4 and comprises a nucleic acid sequence selected from the group consisting of SEQ ID No. 1, SEQ ID No. 3, SEQ ID No. 5, SEQ ID No.7, SEQ ID No. 9, SEQ ID No. 11, SEQ ID No. 13, SEQ ID No. 15, SEQ ID No. 17, SEQ ID No. 19, SEQ ID No. 21 and SEQ ID No. 23; and wherein, a second tropical root-knot resistance providing genomic fragment is selected from the group consisting of SEQ ID No. 25, SEQ ID No. 27, SEQ ID No. 29, SEQ ID No. 31, SEQ ID No. 33, SEQ ID No. 35, SEQ ID No. 37 and SEQ ID No. 39. Preferably, the first tropical root-knot nematode resistance providing genomic fragment on chromosome 4 comprises SEQ ID No. 9; more preferably SEQ ID No. 9 and SEQ ID No. 11; more preferably SEQ ID No. 5, SEQ ID No. 7, SEQ ID No. 9 and SEQ ID No. 11; even more preferably SEQ ID No. 1, SEQ ID No. 3, SEQ ID No. 5, SEQ ID No. 7, SEQ ID No. 9 and SEQ ID No. 11; yet even more preferably SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19 and SEQ ID No. 21; and most preferably SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, and SEQ ID No. 23. The first tropical root-knot resistance providing genomic fragment is not comprised of SEQ ID No. 2, SEQ ID No. 4, SEQ ID No. 6, SEQ, ID No. 8, SEQ ID No. 10, SEQ ID No. 12, SEQ ID No. 14, SEQ ID No. 16, SEQ ID No. 18, SEQ ID No. 20, SEQ ID No. 22, and SEQ ID No. 24. Preferably, the second tropical root-knot nematode resistance providing genomic fragment on chromosome 8 comprises SEQ ID No. 31, more preferably SEQ ID No. 31 and SEQ ID No. 33; more preferably SEQ ID No. 31, SEQ ID No. 33, SEQ ID No. 35 and SEQ ID No. 37; even more preferably SEQ ID No. 31, SEQ ID No. 33, SEQ ID No. 35, SEQ ID No. 37 and SEQ ID No. 39; most preferably SEQ ID No. 25, SEQ ID No. 27, SEQ ID No. 29, SEQ ID No. 31, SEQ ID No. 33, SEQ ID No. 35, SEQ ID No. 37 and SEQ ID No. 39.

Hence, in one preferred embodiment, the tropical root-knot resistant carrot plant comprises SEQ ID No. 9 and SEQ ID No. 31; more preferably SEQ ID No. 9, SEQ ID No. 11, SEQ ID No. 31 and SEQ ID No. 33; more preferably SEQ ID No. 5, SEQ ID No. 7, SEQ ID No. 9, SEQ ID No. 11, SEQ ID No. 31 and SEQ ID No. 33; even more preferably SEQ ID No. 1, SEQ ID No. 3, SEQ ID No. 5, SEQ ID No. 7, SEQ ID No. 9, SEQ ID No. 11, SEQ ID No. 31, SEQ ID No. 33, SEQ ID No. 35 and SEQ ID No. 37; yet even more preferably SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 31, SEQ ID No. 33, SEQ ID No. 35, SEQ ID No. 37 and SEQ ID No. 39; and most preferably SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, SEQ ID No. 35, SEQ ID No. 37 and SEQ ID No. 39.

According to a preferred embodiment of the present invention, the first tropical root-knot nematode resistance providing genomic fragment is obtained, obtainable, or is from a carrot plant of which representative seed has been deposited at NCIMB (National Collections of Industrial, Food and Marine Bacteria; 5 NCIMB Limited, Ferguson Building; Craibstone Estate, Bucksburn Aberdeen, Scotland, AB21 9YA United Kingdom) on 9 Jun. 2021 under deposit number NCIMB 43792.

According to another preferred embodiment, the present carrot plant is cytoplasmic male sterile (CMS).

According to yet another preferred embodiment, the present carrot plant is a hybrid carrot plant, more preferably a sterile hybrid carrot plant and most preferably a male sterile hybrid carrot plant, e.g., cytoplasmic male sterile.

The present invention further relates to a method for identifying a tropical root-knot nematode resistance providing genomic fragment as found in the present carrot plant. The method comprises the steps of isolating or providing genomic nucleic acid of a carrot plant and identifying in the genomic nucleic acid a first tropical root-knot nematode resistance providing genomic fragment on chromosome 4 by detecting a nucleic acid sequence selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 and SEQ ID No. 23.

Preferably, the present method comprises an optional step of further identifying in the genomic nucleic acid a second tropical root-knot nematode resistance providing genomic fragment on chromosome 8 by detecting a nucleic acid sequence selected from the group consisting of SEQ ID No. 25, SEQ ID No. 27, SEQ ID No. 29, SEQ ID No. 31, SEQ ID No. 33, SEQ ID No. 35, SEQ ID No. 37, and SEQ ID No. 39.

Several common genotyping methods exist for detecting a single nuclear polymorphism (SNP) in a genomic sequence, including PCR-based methods, direct hybridization, fragment analysis, and sequencing. An example of a method suitable for detecting a genomic sequence is isolating DNA from available plant material (e.g., from a piece of a leaf from a plant, or a seed), followed by nucleic acid amplification of isolated DNA (e.g., using PCR) and detecting the presence of said genomic sequence (e.g., by sequencing, measuring fluorescence, or visualizing and analysing PCR amplification using agarose gel electrophoresis).

Additionally, the present invention relates to a method for providing a carrot plant of the invention comprising the steps of:
a) obtaining a carrot plant not comprising a first tropical root-knot nematode resistance providing genomic fragment on chromosome 4 of the carrot plant;
b) crossing the carrot plant with a carrot plant comprising a first tropical root-knot nematode resistance providing genomic fragment on chromosome 4 of the carrot plant; obtaining a seed from said cross and germinating the seed to obtain a carrot plant;
c) isolating genomic nucleic acid from the carrot plant and identifying in the genomic nucleic acid a first tropical root-knot nematode resistance providing genomic fragment on chromosome 4 of the carrot plant by detecting a nucleic acid sequence selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 and SEQ ID No. 23.

Preferably, the present method comprises an optional step of further identifying in the genomic nucleic acid a second tropical root-knot nematode resistance providing genomic fragment on chromosome 8 of the carrot plant by detecting a nucleic acid sequence selected from the group consisting of SEQ ID No. 25, SEQ ID No. 27, SEQ ID No. 29, SEQ ID No. 31, SEQ ID No. 33, SEQ ID No. 35, SEQ ID No. 37, and SEQ ID No. 39.

The present invention also relates to a method of producing a carrot plant of the invention comprising the steps of obtaining a carrot plant not comprising a first tropical root-knot nematode resistance providing genomic fragment on chromosome 4 of said carrot plant; introducing in said carrot plant a first tropical root-knot nematode resistance providing genomic fragment as found in a carrot plant of the present invention, wherein said genomic fragment is not introduced by means of an essentially biological process.

Mutagenesis, transformation with Agrobacterium or CRISPR/Cas can be used to introduce a first tropical root-knot nematode resistance providing genomic fragment by means of a non-essentially biological process.

Suitable mutagenesis methods comprise chemical mutagenesis (e.g., using ethyl methanesulfonate (EMS), N-methyl-N-nitrosourea (MNU), N-ethyl-N-nitrosourea (ENU), sodium azide (NaN3), methylnitrosoguanidine (MNNG), diethyl sulfonate (DES), TILLING, or mutagenesis by generating reactive oxygen species) and radiation mutagenesis (e.g., using UV radiation or ion beam radiation). Mutagenesis can lead to one or more mutations located in the coding sequence (mRNA, cDNA or genomic sequence) or in the associated non-coding sequence and/or regulatory sequence regulating the level of expression of the coding sequence. The presence of one or more mutations (e.g., insertion, inversion, deletion and/or replacement of one or more nucleotide(s)) may lead to the encoded protein having a new or altered functionality (gain of function), reduced functionality (reduced function) or no functionality (loss-of-function), e.g., due to the protein being truncated or having an amino acid sequence wherein one or more amino acids are deleted, inserted or replaced. Such changes may lead to the protein having a different 3D structure or conformation, being targeted to a different sub-cellular compartment, having one or more modified catalytic domains, having a modified binding activity to nucleic acids or proteins, etcetera.

Alternatively, the resistance according to the invention can be introduced in a plant cell by transformation (e.g., using Agrobacterium tumefaciens). Genomic fragments can be amplified by long-range PCR amplifications, de novo synthesized, or isolated from gels or columns (e.g., after restriction digestion). The resulting fragments can be reassembled (e.g., in yeast) or introduced in an expression vector, subsequently transformed into carrot plant cells and allowed to integrate or recombine with the carrot plant genome. The fragment may be introduced in a single step or in a series of transformations ultimately resulting in a carrot plant comprising the resistance of the present invention.

Alternatively, the CRISPR/Cas9 system can be used to introduce tropical root-knot nematode resistance in a carrot plant by enabling robust and precise targeted genome modifications, as well as CRISPR-based screening, e.g., using pooled gRNA libraries with complete coverage and distribution over the carrot genome, and whole genome mutagenesis.

According to a second aspect, the present invention also relates to a seed capable of providing a plant of the present invention

Seeds can be coated, coloured, washed, polished, encrusted, pelleted, primed or undergo a combination of treatments. Coated seeds are covered by a relatively thin layer of polymer supplied to the seed; to this polymer fungicides or insecticides can be added to protect the seed against soil borne pathogens and insect damage. Additionally, a dye can be added. This added colour gives the farmer the opportunity to check for correct drilling of the seeds. Alternatively, also other beneficial compounds can be added as micronutrients or beneficial micro-organisms promoting the growth of the young seedlings. Encrusted seeds are not only covered by a polymer with or without extra substances, as described above, but the seeds are provided with a smooth surface as well. This makes drilling easier, and the added weight enables a more precise direct drilling of the seeds. Polishing removes the outermost layer of the seed, so that the seed assumes a more rounded form. Polishing and washing promotes germination of the seed. With pelleting the seeds are covered with more material, e.g., polymer bound clay, to produce a regularly shaped, round pellet. This pellet, besides having protecting substances described above, can be constructed in such a way that it will melt or split after water uptake. Priming or pre-germination is a treatment in which seeds are given enough moisture to initiate germination of the embryo inside the seed. This results in a faster emergence of the seedling, a higher emergence rate and better growth. It is believed that priming leads to a better root system and faster growth.

According to a third aspect, the present invention further relates to a plant cell, a protoplast, a plant organ, plant tissue, edible parts, pollen, microspores, ovaries, ovules, egg cells, callus, suspension culture, somatic embryos, embryos, or plant parts of the present plants resistant to tropical root-knot nematodes. Plant parts include, but are not limited to, the shoot, the stalk, the stem, leaves, blossoms, inflorescence, roots, fruits, and cuttings.

Additionally, the present invention relates to use of a nucleic acid sequence selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 and SEQ ID No. 23 for identifying or providing a first tropical root-knot nematode resistance providing genomic fragment as found in a carrot plant of which representative seed has been deposited under deposit number NCIMB 43792. The indefinite article “a” or “an” can also here be used interchangeably with the phrases “one or more” or “at least one”. As such, the word “a” should be understood to mean one, two, three, four, five, six, or more, or all. The nucleic acid sequences are closely linked to the first tropical root-knot nematode resistance providing genomic fragment and can be used as molecular markers, e.g., in molecular marker-assisted breeding. Molecular marker-assisted breeding greatly increases efficiency and precision of plant breeding, and includes several modern breeding strategies, including marker-assisted selection, marker-assisted backcrossing, marker-assisted recurrent selection, and genome-wide selection or genomic selection.

The present invention also relates to nucleic acid sequences which co-segregate with a first tropical root-knot nematode providing genomic fragment as present in deposit number NCIMB 43792, which nucleic acid sequences are selected from the group consisting of SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, and SEQ ID No. 23.

The present invention will be further detailed in the following examples.

### EXAMPLES

### Example 1: Mutagenesis of Carrot Plants to Introduce Tropical Root-Knot Nematode Resistance

Random mutagenesis followed by forward screening is a useful method for identifying mutant carrot plants with resistance to tropical root-knot nematodes. A mutagenized library can be generated by subjecting seeds to a step of mutagenesis, preferably random mutagenesis. Such a step may comprise, but is not limited to, the treatment of a pool of 100.000 to 200.000 seeds with a chemical mutagen, or a mixture of chemical mutagens, e.g., 0.25% EMS for 16 hours at room temperature; alternatively, radiation can be used (e.g., gamma-radiation from a radioactive Cobalt-60 source). Preferably, only a mildly mutagenized library (fewer than 1% of all genes contain a mutation in a coding region) is generated. Nevertheless, the mutagenesis step will lead to the loss of germination in some seeds. In contrast to irradiation, which can lead to mutations varying from single base substitutions or deletions to large deletions, EMS produces predominantly random point mutations by nucleotide substitution, particularly by guanine alkylation.

The mutagenized seeds can be sown and propagated in a field using standard practices. The mutagenized seed will generate plants that each have a particular set of mutations. Plants can be harvested in pools and viable seeds can be sown again (F1 population). To obtain an F1S1 population seeds can be collected from the F1 plants after selfing. As the mutations will segregate in an FIS1 population, the resistance in this population can be evaluated and used to map the resistance against tropical root-knot nematodes.

### Example 2. General Protocol for Assessing Resistance

Four-week-old carrot plants are transplanted into pots filled with fine white sand. One week after transplanting, plants are tested for tropical root-knot nematode resistance by injecting 1 ml inoculation medium containing 2000-2500 tropical root-knot nematode eggs/ml next to the root system at a depth of 0.5 cm. Plants are not watered for 48 h after inoculation to prevent flushing of tropical root-knot nematode eggs.

Eight to ten weeks after inoculation plants are assessed for severity of tropical root-knot nematode infection. Plants are uprooted and the root system is flushed with tap water to remove the potting soil and fine white sand. After washing, plants are placed in 10% carmine staining solution for five minutes. The number of galls is subsequently counted under a microscope. The maximum number of galls counted per plant is 50. It is carefully checked that the susceptible control plants indeed show the expected symptoms of tropical root-knot nematode infection and that the resistant controls show no infection. Per accession the average number of galls is calculated (AVG), as well as the standard deviation (STD), the percentage of plants with only 2 galls or less (% ≤2) or 5 galls or less (% ≤5), which are all indicative values for the level of resistance.

### Example 3. Results of Assessment for Resistance Against Tropical Root-Knot Nematode Resistance

TABLE 2
Several sources of resistance were tested with different
populations, including sources described in literature.
Mi-WU-1 is a Meloidogyne incognita
population obtained from the nematology group at
Wageningen University. MAU is a Meloidogyne arenaria
population originating from North America.
A minimum of 10 plants were tested per condition.

| | Population | AVG | STD | % ≤2 | % ≤5 |
|---|---|---|---|---|---|
| Sus 1 | Mi-WU-1 | 50.0 | 0.0 | 0% | 0% |
| Sus 2 | Mi-WU-1 | 42.1 | 13.8 | 0% | 0% |
| Res | Mi-WU-1 | 2.4 | 2.8 | 50% | 88% |
| SFF | Mi-WU-1 | 7.3 | 7.6 | 32% | 56% |
| Homs | Mi-WU-1 | 12.5 | 11.4 | 15% | 32% |
| Sus 1 | MAU | 16.0 | 8.5 | 0% | 13% |
| Sus 2 | MAU | 35.6 | 11.2 | 0% | 0% |
| Res | MAU | 0.2 | 0.7 | 94% | 100% |
| SFF | MAU | 0.6 | 0.7 | 100% | 100% |
| Homs | MAU | 0.2 | 0.4 | 100% | 100% |

Explanation of abbreviations used: AVG is the average number of galls counted per plant under a microscope; STD is the standard deviation of the number of galls counted per plant under a microscope; % ≤2 is the percentage of plants that had 2 or fewer galls; % ≤5 is the percentage of plants that had 5 or fewer galls.

### Example 4: Molecular Characterization of Genomic DNA and Mapping of the Resistance Genes

Applying the genetic resource for resistance, here referred to as Res, an F1S1 population was made by crossing the source of resistance to the sus 1 carrot line, after which the resulting F1 plant was self-pollinated.

At least 2000 seeds were harvested from the F1S1 generation of a cross between the distinctive source of resistance and the sus 1 carrot line. To perform a QTL mapping, 240 plants of the cross were tested with the Mi-WU-1 isolate. From each individual plant, leaf material was used for DNA isolation and successive marker analysis.

Using SNP markers covering the entire genome, a QTL was found on chromosome 4 (C4). This QTL is defined by the SNP markers listed in the table below.

TABLE 3
SNPs for the detection of the tropical root-knot nematode resistance
providing genomic fragment on chromosome 4. The genomic position
of each SNP was determined using the published genome
assembly PRJNA268187 Daucus carota Ver2.

| | | | Position on | Allele linked | Alternative |
|---|---|---|---|---|---|
| SNP | ID | Chromosome | chromosome (bp) | to resistance | allele |
| 1 | 9847 | 4 | 10231839 | G | A |
| 2 | 6624 | 4 | 10367643 | C | A |
| 3 | 9848 | 4 | 10375226 | A | G |
| 4 | 9849 | 4 | 10503519 | C | T |
| 5 | 6873 | 4 | 10507269 | C | T |
| 6 | 9850 | 4 | 10627267 | A | G |
| 7 | 6524 | 4 | 10667421 | A | G |
| 8 | 8738 | 4 | 10674185 | T | C |
| 9 | 2281 | 4 | 10705926 | T | G |
| 10 | 8363 | 4 | 10734365 | C | T |
| 11 | 9851 | 4 | 10753940 | T | G |
| 12 | 7147 | 4 | 10957339 | A | G |

TABLE 4
Sequences for the detection of the resistance
against tropical root-knot nematodes on
chromosome 4. The genomic position of each
SNP was determined using the published genome
assembly PRJNA268187 Daucus carota Ver2.
Nucleotide base codes are according to the
International Union of Pure and Applied
Chemistry (IUPAC) code (see Table 5).

| SEQ | | Genomic | |
|---|---|---|---|
| ID | | position | |
| No. | SNP | of SNP* | Nucleic acid sequence |
| 1 | 1 | CHR4_ | TGTTTGAGACTCWACTCTTCTCCTTTACCAACG |
| | | 10231839 | AAAGTATTTATTTGTGA[G]CAATCTTTCATGTCA |
| | | | AACCTAGCAAGTACCTTCTCAATATGGCTCTTTT |
| | | | A |
| 2 | 1 | CHR4_ | TGTTTGAGACTCWACTCTTCTCCTTTACCAACG |
| | | 10231839 | AAAGTATTTATTTGTGA[A]CAATCTTTCATGTCA |
| | | | AACCTAGCAAGTACCTTCTCAATATGGCTCTTTT |
| | | | A |
| 3 | 2 | CHR4_ | TCCTATCTCTCTTCCTCCTCACAATCCTTCCCATT |
| | | 10367643 | CT[C]CACTCCTTACCAGCCATACCAGACCCAAC |
| | | | CACA |
| 4 | 2 | CHR4_ | TCCTATCTCTCTTCCTCCTCACAATCCTTCCCATT |
| | | 10367643 | CT[A]CACTCCTTACCAGCCATACCAGACCCAAC |
| | | | CACA |
| 5 | 3 | CHR4_ | ATTATACTTATACTTGTTTAAATATGTGGTAACT |
| | | 10375226 | TTGGGTGCCAATCACT[A]GAGCAAATTTTTATG |
| | | | TAAAAGTTGTCAAAAATACAAACAAAGRGAGA |
| | | | GAA |
| 6 | 3 | CHR4_ | ATTATACTTATACTTGTTTAAATATGTGGTAACT |
| | | 10375226 | TTGGGTGCCAATCACT[G]GAGCAAATTTTTATG |
| | | | TAAAAGTTGTCAAAAATACAAACAAAGRGAGA |
| | | | GAA |
| 7 | 4 | CHR4_ | CTTCACAACTATTTACHAAGTAATTTAAAACTTC |
| | | 10503519 | TTTAATCTGTGAAGCT[C]GATATCTTATTGCTTT |
| | | | AACTCCTATAACCCGACWTTCTCAACATATTTC |
| | | | T |
| 8 | 4 | CHR4_ | CTTCACAACTATTTACHAAGTAATTTAAAACTTC |
| | | 10503519 | TTTAATCTGTGAAGCT[T]GATATCTTATTGCTTT |
| | | | AACTCCTATAACCCGACWTTCTCAACATATTTC |
| | | | T |
| 9 | 5 | CHR4_ | CGACCAATGATATCAGTTTTTTCGACGATAATA |
| | | 10507269 | ATCT[C]GGGTCGTTAGAATTGGATTCTCTCTGGA |
| | | | CTTGA |
| 10 | 5 | CHR4_ | CGACCAATGATATCAGTTTTTTCGACGATAATA |
| | | 10507269 | ATCT[T]GGGTCGTTAGAATTGGATTCTCTCTGGA |
| | | | CTTGA |
| 11 | 6 | CHR4_ | TTATATACTGTATAAAACAATGATTAGTGCAAC |
| | | 10627267 | AAAAATTAGTGATAGTC[A]TAGTTATAAATACT |
| | | | GGTAAGGAAACTAGTTTTTAGTTTGTATTTAAG |
| | | | AGT |
| 12 | 6 | CHR4_ | TTATATACTGTATAAAACAATGATTAGTGCAAC |
| | | 10627267 | AAAAATTAGTGATAGTC[G]TAGTTATAAATACT |
| | | | GGTAAGGAAACTAGTTTTTAGTTTGTATTTAAG |
| | | | AGT |
| 13 | 7 | CHR4_ | ATCCGTCAATTAGAAAAAGTTAAAAAACACTCT |
| | | 10667421 | CTCT[A]GACACCCATTCTCATTTGAAGCAACAC |
| | | | GTGATG |
| 14 | 7 | CHR4_ | ATCCGTCAATTAGAAAAAGTTAAAAAACACTCT |
| | | 10667421 | CTCT[G]GACACCCATTCTCATTTGAAGCAACAC |
| | | | GTGATG |
| 15 | 8 | CHR4_ | GCTACAACTTTTATCTTTTTCAGTCTTTACATGA |
| | | 10674185 | AGTAMTAGCCGTAAAT[T]TTKCAAGCCAAAGGT |
| | | | GCAACACATTGTAGACTCGACAATATCTTTCAC |
| | | | TC |
| 16 | 8 | CHR4_ | GCTACAACTTTTATCTTTTTCAGTCTTTACATGA |
| | | 10674185 | AGTAMTAGCCGTAAAT[C]TTKCAAGCCAAAGGT |
| | | | GCAACACATTGTAGACTCGACAATATCTTTCAC |
| | | | TC |
| 17 | 9 | CHR4_ | CACTTGTCCTGAAGAATGTCAAGGCAAATATTC |
| | | 10705926 | CCATACTGGTCAACATT[T]GGATGGAAGCACAT |
| | | | TGTCTCAAACTTCACTTGGGGAGGCTTGAAAGG |
| | | | ATA |
| 18 | 9 | CHR4_ | CACTTGTCCTGAAGAATGTCAAGGCAAATATTC |
| | | 10705926 | CCATACTGGTCAACATT[G]GGATGGAAGCACAT |
| | | | TGTCTCAAACTTCACTTGGGGAGGCTTGAAAGG |
| | | | ATA |
| 19 | 10 | CHR4_ | TAAGGCTTGCRTTGGMTTAAARAGTTGCAGCAT |
| | | 10734365 | CGACGTATCAGTGTCAA[C]TTTTGGGAATCCGT |
| | | | GTAGAGGAGTTACAAAGAGTTTAGCAGTAGAA |
| | | | GCWT |
| 20 | 10 | CHR4_ | TAAGGCTTGCRTTGGMTTAAARAGTTGCAGCAT |
| | | 10734365 | CGACGTATCAGTGTCAA[T]TTTTGGGAATCCGT |
| | | | GTAGAGGAGTTACAAAGAGTTTAGCAGTAGAA |
| | | | GCWT |
| 21 | 11 | CHR4_ | ATGACACCAACACGGTGAAAGTCAGTGTATCTG |
| | | 10753940 | GCCTTTTGGAGGTGGAT[T]TGAAGGTGACTCCA |
| | | | ATAAAAGAAAAGGAAAACAAGGTGCACAACTA |
| | | | CCAG |
| 22 | 11 | CHR4_ | ATGACACCAACACGGTGAAAGTCAGTGTATCTG |
| | | 10753940 | GCCTTTTGGAGGTGGAT[G]TGAAGGTGACTCCA |
| | | | ATAAAAGAAAAGGAAAACAAGGTGCACAACTA |
| | | | CCAG |
| 23 | 12 | CHR4_ | CCATACCCATTCTTTTGAAATGAAAATGCAAAA |
| | | 10957339 | CAGA[A]GTCTTCCAGTAGCTCTCTACCTGCATTT |
| | | | GACAA |
| 24 | 12 | CHR4_ | CCATACCCATTCTTTTGAAATGAAAATGCAAAA |
| | | 10957339 | CAGA[G]GTCTTCCAGTAGCTCTCTACCTGCATTT |
| | | | GACAA |

*CHR4 = chromosome 4

TABLE 5
Nucleotide base codes according to the
International Union of Pure and
Applied Chemistry (IUPAC) code.

| Symbol | Nucleotide Base |
|---|---|
| A | Adenine |
| C | Cytosine |
| G | Guanine |
| T | Thymine |
| N | A or C or G or T |
| M | A or C |
| R | A or G |
| W | A or T |
| S | C or G |
| Y | C or T |
| K | G or T |
| V | Not T |
| H | Not G |
| D | Nor C |
| B | Not A |

### Example 5: QTL on C4 is Not Allelic to Resistance on Chromosome 4 Previously Discovered in SFF

Several individual plants from the SPF line were used to make crosses with the sus 1 carrot line. These individual plants were also self-pollinated. The progeny was tested for resistance. The selfings of the individual plants from SFF still show a level of resistance, while all hybrids are fully susceptible.

In contrast, when similar crosses were made with Res plants, the progeny of the selfings showed a high level of resistance. Also, hybrids made by crossing with the susceptible sus 1 and sus 2 carrot lines still showed a level of resistance.

From these tests, it is clear that the previously described QTL on C4 derived from SFF does not provide any level of resistance when present in a heterozygous form, in contrast to the QTL derived from Res.

TABLE 6
Several selfings of SFF and Res as well as crosses with susceptible
carrot lines were tested with a Meloidogyne incognita population
obtained from the nematology group at Wageningen
University (Mi-WU-1).

| Name | # plants | AVG | STD | % ≤2 | % ≤5 |
|---|---|---|---|---|---|
| Res*Res | 6 | 1.8 | 0.8 | 83% | 100% |
| Sus 1*Sus 1 | 8 | 50.0 | 0.0 | 0% | 0% |
| Sus 2*Sus 2 | 8 | 42.1 | 13.8 | 0% | 0% |
| Sus 1*Res (C4) | 235 | 15.1 | 9.6 | 2% | 15% |
| SFF*SFF | 10 | 8.0 | 4.1 | 10% | 20% |
| Sus 1*SFF | 14 | 50.0 | 0.0 | 0% | 0% |

Explanation of abbreviations used: #plants is the number of plants tested, AVG is the average number of galls counted per plant under a microscope; STD is the standard deviation of the number of galls counted per plant under a microscope; % ≤2 is the percentage of plants that had 2 or fewer galls; % ≤5 is the percentage of plants that had 5 or fewer galls.

### Example 6: QTL on C4 can be Combined With QTL on C8 to Further Enhance Resistance

Several resistances against Meloidogyne javanica and Meloidogyne incognita have been previously described in literature (e.g., Parsons et al. 2015). As the QTL on chromosome 8 (C8), referred to as Mj-1, has been described as a monogenic dominant trait that imparts resistance to Meloidogyne javanica and a partial resistance, plants carrying a tropical root-knot nematode resistance providing genomic fragment on C4 and C8 were combined and tested for resistance to determine whether this provided an even more robust resistance to tropical root-knot nematodes.

The tests showed that the resistance against tropical root-knot nematodes was further improved by combining the QTLs on C4 and C8.

TABLE 7
Carrot plants with a QTL on C4, C4 and C8 and only C8 were tested for
root-knot nematode resistance. Mi-WU-1 is a Meloidogyne incognita
population obtained from the nematology group at Wageningen University.
MAU is a Meloidogyne arenaria population originating
from North America.

| ID | Locus | Isolate | #plants | AVG | STD | % ≤ 2 | % ≤ 5 |
|---|---|---|---|---|---|---|---|
| Y9380 | C4 | Mi-WU-1 | 89 | 0.8 | 2.9 | 93 | 99 |
| Y9381 | C4, C8 | Mi-WU-1 | 24 | 0.1 | 0.4 | 100 | 100 |
| Y9383 | C8 | Mi-WU-1 | 176 | 23.0 | 16.1 | 4 | 10 |
| Y9380 | C4 | MAU | 68 | 1.9 | 3.7 | 74 | 97 |
| Y9381 | C4, C8 | MAU | 18 | 0.2 | 0.7 | 95 | 100 |
| Y9383 | C8 | MAU | 176 | 7.0 | 9.3 | 42 | 61 |

Explanation of abbreviations used: #plants is the number of plants tested, AVG is the average number of galls counted per plant under a microscope; STD is the standard deviation of the number of galls counted per plant under a microscope; % ≤2 is the percentage of plants that had 2 or fewer galls; % ≤5 is the percentage of plants that had 5 or fewer galls

SNP markers were used to detect the tropical root-knot resistance providing genomic fragment on C8 and to identify and provide plants carrying this resistance providing fragment.

TABLE 8
SNPs for the detection of the tropical root-knot nematode resistance
providing genomic fragment on chromosome 8. The genomic
position of each SNP was determined using the published
genome assembly PRJNA268187 Daucus carota Ver2.

| | | | Position on | Allele linked | Alternative |
|---|---|---|---|---|---|
| SNP | ID | Chromosome | chromosome (bp) | to resistance | allele |
| 13 | 7136 | 8 | 17676345 | C | G |
| 14 | 6692 | 8 | 17781631 | G | A |
| 15 | 4421 | 8 | 17968674 | C | A |
| 16 | 6419 | 8 | 18836406 | G | C |
| 17 | 5095 | 8 | 21101599 | T | C |
| 18 | 4255 | 8 | 21235813 | A | G |
| 19 | 5020 | 8 | 21254357 | T | C |
| 20 | 4441 | 8 | 21422127 | C | T |

TABLE 9
Sequences for the detection of the resistance
against tropical root-knot nematodes on
chromosome 8. The genomic position of each
SNP was determined using the published genome
assembly PRJNA268187 Daucus carota Ver2.
Nucleotide base codes are according to the
International Union of Pure and Applied
Chemistry (IUPAC) code (see Table 5).

| SEQ | | Genomic | |
|---|---|---|---|
| ID | | position | |
| No. | SNP | of SNP* | Nucleic acid sequence |
| 25 | 13 | CHR8_ | AAACATTAAACTTGGCGGATGATGATATATATA |
| | | 17676345 | ATGC[C]GTGCTACTTGTTGCAGCTGCTTATAATC |
| | | | ACTTT |
| 26 | 13 | CHR8_ | AAACATTAAACTTGGCGGATGATGATATATATA |
| | | 17676345 | ATGC[G]GTGCTACTTGTTGCAGCTGCTTATAATC |
| | | | ACTTT |
| 27 | 14 | CHR8_ | GATCGAAGGGGGTGCGGCCAGAGGTCTTTGCCC |
| | | 17781631 | GAGC[G]TATGCCCGTGTCTTCTCCTCGTTACTTG |
| | | | TCCTT |
| 28 | 14 | CHR8_ | GATCGAAGGGGGTGCGGCCAGAGGTCTTTGCCC |
| | | 17781631 | GAGC[A]TATGCCCGTGTCTTCTCCTCGTTACTTG |
| | | | TCCTT |
| 29 | 15 | CHR8_ | CTCTCGGRACCCAACCAATTACCCTACGAACTT |
| | | 17968674 | TCTC[C]TCGAAGAGTACTACACATGTTTTTGCTG |
| | | | CCTCA |
| 30 | 15 | CHR8_ | CTCTCGGRACCCAACCAATTACCCTACGAACTT |
| | | 17968674 | TCTC[A]TCGAAGAGTACTACACATGTTTTTGCTG |
| | | | CCTCA |
| 31 | 16 | CHR8_ | GAGAATCTGCCGTACGGCGTGTTCAAGCCTGAC |
| | | 18836406 | GAAT[G]TTCGGCACCTCGACCTGGCGTTGCTAT |
| | | | CGGRGA |
| 32 | 16 | CHR8_ | GAGAATCTGCCGTACGGCGTGTTCAAGCCTGAC |
| | | 18836406 | GAAT[C]TTCGGCACCTCGACCTGGCGTTGCTAT |
| | | | CGGRGA |
| 33 | 17 | CHR8_ | GCCAAAGGCTACTACAAAGTGGTTGCAAGTTTC |
| | | 21101599 | AAAA[T]TGATGCACAAGGGTGCTGTGTCAAGTG |
| | | | AAGATC |
| 34 | 17 | CHR8_ | GCCAAAGGCTACTACAAAGTGGTTGCAAGTTTC |
| | | 21101599 | AAAA[C]TGATGCACAAGGGTGCTGTGTCAAGTG |
| | | | AAGATC |
| 35 | 18 | CHR8_ | GAAAATATCATCGCCACGAGATAATTTTTGCTG |
| | | 21235813 | ATCA[A]CAAAGACTTGAGAGTAACATTCGACTT |
| | | | CCCGTG |
| 36 | 18 | CHR8_ | GAAAATATCATCGCCACGAGATAATTTTTGCTG |
| | | 21235813 | ATCA[G]CAAAGACTTGAGAGTAACATTCGACTT |
| | | | CCCGTG |
| 37 | 19 | CHR8_ | TTTTACATAGTTACAATTAGAAATTCAGATCCC |
| | | 21254357 | ACCT[T]TTAGACTCCAACCTCTCCGGCTAACTTG |
| | | | AAAGG |
| 38 | 19 | CHR8_ | TTTTACATAGTTACAATTAGAAATTCAGATCCC |
| | | 21254357 | ACCT[C]TTAGACTCCAACCTCTCCGGCTAACTTG |
| | | | AAAGG |
| 39 | 20 | CHR8_ | TACAACTCGATGCTTATATATCACGAAAAGAAC |
| | | 21422127 | TCTT[C]AAGGCAGAAGCAAAATATTGGAATGAA |
| | | | ATAATA |
| 40 | 20 | CHR8_ | TACAACTCGATGCTTATATATCACGAAAAGAAC |
| | | 21422127 | TCTT[T]AAGGCAGAAGCAAAATATTGGAATGAA |
| | | | ATAATA |

*CHR8 = chromosome 8

### Example 7: Introduction of Tropical Root-Knot Resistance Into a Carrot Plant Using Agrobacterium

Transformation of plants using the Agrobacterium tumefaciens system is commonly used to generate plants resistant to pathogens by introducing resistance genes in plants. The Agrobacterium tumefaciens system can also be used to introduce a tropical root-knot resistance providing genomic fragment according to the present invention. Such a fragment is isolated from gels or columns after restriction digestion of a carrot plant comprising a first tropical root-knot nematode resistance providing genomic fragment. The digested fragment can be introduced in a single step or in a series of transformations using Agrobacterium tumefaciens into carrot plant cells. After transformation, stable transformants comprising the resistance of the present invention will be selected by performing a disease test or by determining the presence of the resistance providing genomic fragment using molecular markers.

### Deposit Information

Seed samples of the sources of resistance mentioned above have been deposited at the NCIMB (Ferguson Building, Craibstone Estate, Bucksburn, Aberdeen, Scotland, AB21 9YA) on 9 Jun. 2021 as NCIMB 43792.

1. A carrot plant resistant to tropical root-knot nematodes, comprising on chromosome 4 of said carrot plant a first tropical root-knot nematode resistance providing genomic fragment, wherein said first genomic fragment comprises a nucleic acid sequence selected from the group consisting of SEQ ID Nos. 1, 3, 5, 7, 9, 11, 13, 15, 17, 19, 21 and 23.

2. The carrot plant according to claim 1, further comprising a second tropical root-knot nematode resistance providing genomic fragment on chromosome 8 of the carrot plant, wherein said second genomic fragment comprises a nucleic acid sequence selected from the group consisting of SEQ ID Nos. 25, 27, 29, 31, 33, 35, 37 and 39.

3. The carrot plant according to claim 1 wherein a first tropical root-knot nematode resistance providing genomic fragment is obtained, obtainable, or is from a carrot plant of which representative seed are deposited under deposit number NCIMB 43792.

4. The carrot plant according to claim 1, wherein said carrot plant is cytoplasmic male sterile (CMS).

5. The carrot plant according to claim 1, wherein said carrot plant is a hybrid plant.

6. The carrot plant according to claim 1, wherein said carrot plant is a carrot plant of which representative seed are deposited under deposit number NCIMB 43792.

7. A method for identifying a tropical root-knot nematode resistance providing genomic fragment comprising the steps of:
a) isolating or providing a genomic nucleic acid of a carrot plant;
b) identifying in said genomic nucleic acid a first tropical root-knot nematode resistance providing genomic fragment on chromosome 4 by detecting a nucleic acid sequence selected from the group consisting of SEQ ID Nos. 1, 3, 5, 7, 9, 11, 13, 15, 17, 19, 21 and 23; and
c) optionally, further identifying in said genomic nucleic acid a second tropical root-knot nematode resistance providing genomic fragment on chromosome 8 by detecting a nucleic acid sequence selected from the group consisting of SEQ ID Nos. 25, 27, 29, 31, 33, 35, 37 and 39.

8. A method for providing a tropical root-knot nematode resistant carrot plant comprising the steps of:
a) obtaining a carrot plant not comprising a first tropical root-knot nematode resistance providing genomic fragment on chromosome 4 of said carrot plant;
b) crossing said carrot plant with a carrot plant according to claim 1;
c) obtaining a seed from said cross and germinating said seed to obtain a carrot plant;
d) isolating genomic DNA from said carrot plant and identifying in the genome of said carrot plant a nucleic acid sequence selected from the group consisting of SEQ ID Nos. 1, 3, 5, 7, 9, 11, 13, 15, 17, 19, 21 and 23; and
e) optionally, further identifying in the genome of said carrot plant a nucleic acid sequence selected from the group consisting of SEQ ID Nos. 25, 27, 29, 31, 33, 35, 37, and 39.

9. A method for providing a tropical root-knot nematode resistant carrot plant, comprising the steps of:
a) obtaining a carrot plant not comprising a first tropical root-knot nematode resistance providing genomic fragment on chromosome 4 of said carrot plant; and
b) introducing in said carrot plant a first tropical root-knot nematode resistance providing genomic fragment as found in a carrot plant of which representative seed has been deposited under deposit number NCIMB 43792, wherein said genomic fragment is not introduced by means of an essentially biological process

10. The method according to claim 9, wherein said method for providing comprises the step of mutagenesis, transformation with Agrobacterium or CRISPR/Cas.

11. A seed of the carrot plant according to claim 1, comprising a first tropical root-knot nematode resistance providing genomic fragment wherein said first genomic fragment comprises a nucleic acid sequence selected from the group consisting of SEQ ID Nos. 1, 3, 5, 7, 9, 11, 13, 15, 17, 19, 21 and 23.

12. The seed according to claim 11, wherein said seed is polished, coated, encrusted, pelleted or primed.

13. A plant part, callus, suspension culture, or clone of a carrot plant according to claim 1.

14. (canceled)

15. (canceled)

16. The seed of claim 11, wherein the seed further comprises a second tropical root-knot nematode resistance providing genomic fragment wherein said second genomic fragment comprises a nucleic acid sequence selected from the group consisting of SEQ ID Nos. 25, 27, 29, 31, 33, 35, 37, and 39.
